(19) United States
(12) Patent Application Publication
(10) Pub. No.: US 2024/0408018 A1
(43) Pub. Date: Dec. 12, 2024

(54) PREPARATION METHOD AND APPLICATION METHOD OF INJECTABLE PLA MICROSPHERES

(71) Applicants: eSUNMed Biotechnology (Shenzhen) Co., Ltd., Shenzhen (CN); Shenzhen Esun Industrial Co., Ltd., Shenzhen (CN)

(72) Inventors: Rui CHEN, Shenzhen (CN); Jiankang ZHOU, Shenzhen (CN); Qing XIE, Shenzhen (CN); Yihu YANG, Shenzhen (CN)

(21) Appl. No.: 18/430,682

(22) Filed: Feb. 2, 2024

(30) Foreign Application Priority Data
Jun. 7, 2023 (CN) 2023106697668

(51) Int. Cl. A61K 9/16; A61K 9/00; B01J 13/04

(52) U.S. Cl. CPC A61K 9/1694; A61K 9/0019; A61K 9/1647; B01J 13/043

(57) ABSTRACT

A preparation method and an application method of injectable polylactic acid (PLA) microspheres are provided, which relate to the field of biomedical technologies. The preparation method of the injectable PLA microspheres include: S01, preparing a spray solution; S02, performing homogeneous emulsification on the spray solution to obtain an emulsified spray solution; and S03, drying the emulsified spray solution. The spray solution includes an oil phase and an aqueous phase, and a weight ratio of the oil phase and the aqueous phase is 1-10:1-10. The oil phase includes PLA and an organic solvent, and the aqueous phase includes a surfactant, macromolecular substances and deionized water. A particle size of the injectable PLA microspheres is basically below 60 μm, and the injectable PLA microspheres have a complete form, are independent of each other, do not exhibit agglomeration and have good biocompatibility, which can satisfy the requirement of the injection.

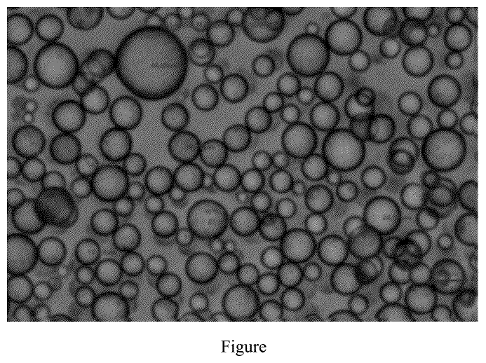

### TECHNICAL FIELD

The disclosure relates to the field of biomedical technologies, and more particularly to a preparation method and an application method of injectable polylactic acid (PLA) microspheres.

### BACKGROUND

In recent years, biodegradable polymer materials come into vision of people, such materials have no toxicity and rejection to a human body, and can be excreted from the human body after being gradually degraded with human metabolism. Due to its great biocompatibility and biodegradability, polylactic acid (PLA) microspheres have been used as a carrier for peptide and protein drugs, and are widely applied in numerous fields such as immunology, gene therapy, tumor therapy and ophthalmology.

Injectable microspheres have been widely used in clinical practice. For the injectable microspheres, they are gradually gradated in the human body after injection, so that a drug loaded on the microspheres is slowly released at a certain rate to maintain a plasma concentration of a lesion site is maintained, so as to extend a half-life period of the drug, and achieve long-term sustained release. For patients, a frequency of administration can be fully reduced to improve compliance. Medical injectable PLA microspheres have unlimited application possibilities with a vigorous development of consumer medical market.

The PLA microspheres can be also applied in a field of medical cosmetology. The medical cosmetology industry has developed rapidly in recent years, upstream filling materials are currently in an upgrade and iteration period, which is updated from hyaluronic acid to recycled materials such as collagen, PLA and poly-L-lactic acid (PLLA). The PLA is a polymer material synthesized by chemistry, which can achieve high purity, and has no immunogen hidden risk. A medical cosmetology filler (also referred to as dermal filler) developed based on the PLA can stimulate an immune system in the human body, and affect immune cells, so as to induce fibroblasts to up-regulate collagen secretion, and promote regeneration and repair of skin tissues.

However, in actual production, the prepared PLA microspheres are prone to agglomeration, and have poor dispersibility in water, thus, the prepared PLA microspheres are difficult to be injected. Meanwhile, a degradation rate of a single PLA microsphere is slow in an early stage, and accelerates in a later stage, which is not conducive to control of drug dosage.

### SUMMARY

Based on problems exist in the related art, the disclosure provides a preparation method and an application method of injectable polylactic acid (PLA) microspheres. The PLA microspheres prepared by the method of the disclosure have a complete microsphere form and a good biocompatibility, which can satisfy requirements of injection.

The disclosure is implemented through the following technical solutions.

Specifically, a preparation method of injectable PLA microspheres is provided, and the preparation method includes:
S01, preparing a spray solution;
S02, performing homogeneous emulsification on the spray solution to obtain an emulsified spray solution; and
S03, drying the emulsified spray solution to obtain the injectable PLA microspheres.

In an embodiment, in the step S01, the spray solution includes an oil phase and an aqueous phase, and a weight ratio of the oil phase and the aqueous phase is 1-10:1-10; and the oil phase includes PLA and an organic solvent, and the aqueous phase includes a surfactant, macromolecular substances and deionized water.

In an embodiment, a molecular weight of the PLA is in a range of 50000-95000.

In an embodiment, the organic solvent is one selected from the group consisting of dichloromethane, trichloromethane and ethyl acetate.

In an embodiment, a concentration of the PLA is in a range of 20-60 grams per liter (g/L).

In an embodiment, the surfactant is one selected from the group consisting of sodium dodecylbenzene sulfonate, polysorbate 80 (also referred to as TWEEN® 80), and polysorbate 20 (also referred to as TWEEN® 20).

The macromolecular substances are a mixture of sodium alginate, whey protein and β-glucose, and a weight ratio of the sodium alginate: the whey protein: the β-glucose is 1-6:1-3:1-5.

In an embodiment, a concentration of the surfactant is in a range of 5-10 g/L, and a concentration of the macromolecular substances is in a range of 0.5-1 g/L.

In an embodiment, in the step S02, a temperature of the homogeneous emulsification for the spray solution is 50 Celsius degrees (° C.), a rotational speed of a homogenizer is in a range of 3000-5000 revolutions per minute (rpm), and a time of the homogeneous emulsification for the spray solution is in a range of 5-10 minutes (min).

In an embodiment, in the step S03, process conditions of the drying the emulsified spray solution are as follows. An input rate of the emulsified spray solution is in a range of 4-20 milliliters per minute (mL/min), a pressure of compressed air is in a range of 0.3-0.5 megapascals (MPa), an air inlet temperature is 160° C., an air outlet temperature is 80° C., and carrier gas is inert gas or air. The PLA microspheres are collected in a cyclone separator, and are dried under reduced pressure below 60° C.

In an embodiment, an application method of the injectable PLA microspheres is provided, and the application method includes:
preparing drug sustained-release microspheres by applying the injectable PLA microspheres; where weight of the drug sustained-release microspheres is in a range of 0.1-10% of weight of the PLA microspheres.

### Beneficial Effects of the Disclosure are as Follows.

(1) The PLA microspheres prepared by a spray drying method adopted by the disclosure is prone to control particle sizes and morphology. The method has a high production rate and can be mass-produced. The particle size of the injectable PLA microspheres is basically below 60 microns (μm), and the PLA microspheres have complete morphology, are independent of each other, do not exhibit agglomeration and have good biocompatibility, which can satisfy the requirements of the injection. The PLA microspheres can be dispersed in water, and the slurry shows a good dispersibility after stewing. The PLA microspheres can be evenly dispersed in water to form even dispersion liquid, instead of floating on an upper layer or depositing on a lower layer of the water.
(2) A degradation weight loss rate of the PLA microspheres prepared in the disclosure keeps a constant rate, which is suitable for the preparation of drug sustained-release microspheres, avoiding the slow degradation in the early stage and slow drug release, rapid degradation in the later stage and uncontrollable drug dosage caused by rapid drug release in traditional single PLA microsphere.

### BRIEF DESCRIPTION OF DRAWING

Drawing is used to provide a further description of the disclosure, and is part of the specification, the drawing is combined with embodiments to describe the disclosure, and the drawing does not limit the disclosure.

FIGURE illustrates an optical microscopic diagram of injectable polylactic acid (PLA) microspheres according to an embodiment 1 of the disclosure.

### DETAILED DESCRIPTION OF EMBODIMENTS

Technical solutions of the disclosure are further elaborated in conjunction with embodiments below, however, a scope of protection of the disclosure is not limited by the following embodiments.

In the embodiments and comparative embodiments of the disclosure, a molecular weight of polylactic acid (PLA) is 5000.

### Embodiment 1

Injectable PLA microspheres are prepared by the following steps S01-S03.

In step S01, a spray solution is prepared.

The spray solution includes an oil phase and an aqueous phase, and a weight ratio of the oil phase and the aqueous phase is 5:3.

The oil phase includes PLA and dichloromethane, and a concentration of the PLA is 40 grams per liter (g/L).

The aqueous phase includes polysorbate 20, macromolecular substances and deionized water. A concentration of the polysorbate 20 is 5 g/L, and a concentration of the macromolecular substances is 0.5 g/L.

The macromolecular substances are a mixture of sodium alginate, whey protein and β-glucose, and a weight ratio of the sodium alginate: the whey protein: the β-glucose is 2:1:1.

In step S02, homogeneous emulsification is performed on the spray solution to obtain an emulsified spray solution.

A temperature of the homogeneous emulsification for the spray solution is 50 Celsius degrees (° C.), a rotational speed of a homogenizer is 4000 revolutions per minute (rpm), and a time of the homogeneous emulsification for the spray solution is 10 minutes (min).

In step S03, the emulsified spray solution is dried to obtain the PLA microspheres.

An input rate of the emulsified spray solution is 10 milliliters per minute (mL/min), a pressure of compressed air is 0.5 megapascals (MPa), an air inlet temperature is 160° C., an air outlet temperature is 80° C., and carrier gas is air. The PLA microspheres are collected in a cyclone separator, and are dried under reduced pressure below 60° C.

The PLA microspheres of the embodiment 1 is dispersed in water to obtain dispersion liquid. After mixing, water in the dispersion liquid is dried to obtain dried PLA microspheres, the dried PLA microspheres are observed by an optical microscope, and a result is shown in FIGURE. FIGURE illustrates a microscopic diagram of the injectable PLA microspheres according to the embodiment 1 of the disclosure. It can be seen from FIGURE, the PLA microspheres have complete forms, are independent of each other, do not exhibit agglomeration and have good dispersibility.

### Embodiment 2

Injectable PLA microspheres are prepared by the following steps S01-S03.

In step S01, a spray solution is prepared.

The spray solution includes an oil phase and an aqueous phase, and a weight ratio of the oil phase and the aqueous phase is 3:5.

The oil phase includes PLA and dichloromethane, and a concentration of the PLA is 50 g/L.

The aqueous phase includes sodium dodecylbenzene sulfonate, macromolecular substances and deionized water. A concentration of the sodium dodecylbenzene sulfonate is 5 g/L, and a concentration of the macromolecular substances is 0.5 g/L.

The macromolecular substances are a mixture of sodium alginate, whey protein and β-glucose, and a weight ratio of the sodium alginate: the whey protein: the β-glucose is 3:1:2.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 mL/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Embodiment 3

Injectable PLA microspheres are prepared by the following steps S01-S03.

In step S01, a spray solution is prepared.

The spray solution includes an oil phase and an aqueous phase, and a weight ratio of the oil phase and the aqueous phase is 5:5.

The oil phase includes PLA and dichloromethane, and a concentration of the PLA is 40 g/L.

The aqueous phase includes polysorbate 80, macromolecular substances and deionized water. A concentration of the polysorbate 80 is 5 g/L, and a concentration of the macromolecular substances is 0.5 g/L.

The macromolecular substances are a mixture of sodium alginate, whey protein and β-glucose, and a weight ratio of the sodium alginate: the whey protein: the β-glucose is 6:3:5.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 mL/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Comparative Embodiment 1

Injectable PLA microspheres are prepared by the following steps S01-S03.

In step S01, a spray solution is prepared.

The spray solution includes PLA and dichloromethane, and a concentration of the PLA is 40 g/L.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 ml/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Comparative Embodiment 2

Injectable PLA microspheres are prepared by the following steps S01-S03.

In step S01, a spray solution is prepared.

The spray solution includes an oil phase and an aqueous phase, and a weight ratio of the oil phase and the aqueous phase is 5:3.

The oil phase includes PLA and dichloromethane, and a concentration of the PLA is 40 g/L.

The aqueous phase includes polysorbate 20 and deionized water, and a concentration of the polysorbate 20 is 5 g/L.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 mL/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Comparative Embodiment 3

Injectable PLA microspheres are prepared by the following steps S01-S03.

In step S01, a spray solution is prepared.

The spray solution includes an oil phase and an aqueous phase, and a weight ratio of the oil phase and the aqueous phase is 5:3.

The oil phase includes PLA and dichloromethane, and a concentration of the PLA is 40 g/L.

The aqueous phase includes polysorbate 20, macromolecular substances and deionized water. A concentration of the polysorbate 20 is 5 g/L, and a concentration of the macromolecular substances is 0.5 g/L.

The macromolecular substances are a mixture of sodium alginate and β-glucose, and a weight ratio of the sodium alginate and the β-glucose is 2:1.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 mL/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Comparative Embodiment 4

Injectable PLA microspheres are prepared by the following steps S01-S03.

In step S01, a spray solution is prepared.

The spray solution includes an oil phase and an aqueous phase, and a weight ratio of the oil phase and the aqueous phase is 5:3.

The oil phase includes PLA and dichloromethane, and a concentration of the PLA is 40 g/L.

The aqueous phase includes polysorbate 20, macromolecular substances and deionized water. A concentration of the polysorbate 20 is 5 g/L, and a concentration of the macromolecular substances is 0.5 g/L.

The macromolecular substances are a mixture of whey protein and β-glucose, and a weight ratio of the whey protein and the β-glucose is 1:1.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 mL/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Comparative Embodiment 5

Injectable PLA microspheres are prepared by the following steps S01-S03.

In step S01, a spray solution is prepared.

The spray solution includes an oil phase and an aqueous phase, and a weight ratio of the oil phase and the aqueous phase is 5:3.

The oil phase includes PLA and dichloromethane, and a concentration of the PLA is 40 g/L.

The aqueous phase includes the polysorbate 20, macromolecular substances and deionized water. A concentration of the polysorbate 20 is 5 g/L, and a concentration of the macromolecular substances is 0.5 g/L.

The macromolecular substances are a mixture of sodium alginate and whey protein, and a weight ratio of the sodium alginate and the whey protein is 2:1.

In step S02, homogeneous emulsification is performed on the spray solution to obtain an emulsified spray solution.

A temperature of the homogeneous emulsification for the spray solution is 50° C., a rotational speed of the homogenizer is 4000 rpm, and a time of the homogeneous emulsification for the spray solution is 10 min.

In step S03, the emulsified spray solution is dried to obtain the PLA microspheres.

An input rate of the emulsified spray solution is 10 mL/min, a pressure of the compressed air is 0.5 MPa, an air inlet temperature is 160° C., an air outlet temperature is 80° C., and the carrier gas is air. The PLA microspheres are collected in the cyclone separator, and are dried under reduced pressure below 60° C.

### Experimental Embodiment 1

Comparison of weight-loss rate of the PLA microspheres during degradation is as follows.

The PLA microspheres of embodiments 1-3 and comparative embodiments 1-5 are added into pH=7.4 buffer solutions to obtain mixtures, respectively, and a degradation experiment is performed on the mixtures in a shaker with a temperature of 37° C. The mixtures are taken out at certain intervals, and are respectively centrifuged, cleaned and dried to obtain weight of residual microspheres, the results are shown in Table 1.

TABLE 1

| | First | Second | Third | Fourth | Fifth | Sixth |
|---|---|---|---|---|---|---|
| Groups | month | month | month | month | month | month |
| Embodiment 1 | 92.6 | 85.4 | 61.3 | 43.5 | 33.1 | 21.8 |
| Embodiment 2 | 93.4 | 85.8 | 60.5 | 42.0 | 34.9 | 23.7 |
| Embodiment 3 | 91.7 | 84.2 | 62.3 | 44.1 | 33.7 | 21.0 |
| Comparative | 95.1 | 92.9 | 90.2 | 86.7 | 66.3 | 43.2 |
| embodiment 1 | | | | | | |
| Comparative | 93.4 | 91.7 | 88.5 | 84.1 | 63.0 | 39.5 |
| embodiment 2 | | | | | | |
| Comparative | 92.5 | 88.4 | 70.9 | 59.4 | 35.8 | 21.6 |
| embodiment 3 | | | | | | |
| Comparative | 93.2 | 86.9 | 71.8 | 55.7 | 34.6 | 19.5 |
| embodiment 4 | | | | | | |
| Comparative | 92.9 | 87.4 | 72.5 | 58.4 | 36.9 | 20.8 |
| embodiment 5 | | | | | | |

Values in Table 1 are weight percentages of the residual microspheres. As shown in Table 1, the degradation rate of the microspheres of the comparative embodiment 1 is slow in the early stage, the microspheres of the comparative embodiment 1 are only degraded by 10% in the third month, and the degradation rate of the microspheres significantly increase from the fourth month to the sixth month. Therefore, it is difficult to control the drug release amount when the microspheres of the comparative embodiment 1 are used to prepare the drug sustained-release microspheres. Similarly, the degradation rate is slow in the early stage and is significantly increased in the later stage in the comparative embodiment 2. In the comparative embodiments 4 and the comparative embodiment 5, the macromolecular substances are added, and the macromolecular substances can improve the degradation rate of the microspheres in the early stage, but the degradation rate in the early and later stages is not kept at a stable level. In the embodiments 1-3 of the disclosure, three kinds of macromolecular substances are mixed to prepare the PLA microspheres, the obtained microspheres can keep a stable degradation rate, which solves the unstable situation that less degradation in the early stage and more degradation in the later stage, and can ensure a stable drug release rate when the microspheres of the embodiments 1-3 are used for preparing drug sustained-release microspheres.

### Experimental Embodiment 2

Absorbability comparison of the PLA microspheres is as follows.

A disposable syringe with a volume of 1 milliliter (mL) equipped with a needle with an inner diameter of 0.5 millimeters (mm) and a length of 19.7 mm (i.e., a commonly used syringe gun head with a volume of 1 mL) is used as an injection rate detection device, and 1 mL of microsphere dispersion liquid (i.e., a dry weight of the microspheres is 0.1 g, and is recorded as M0) is used as an experimental sample. 0.1 g of the microspheres of the embodiments 1-3 and the comparative embodiments 1-5 are respectively weighed and dispersed in 1 mL of distilled water to obtain mixtures, and then placed into glass bottles, and microsphere dispersion liquids are obtained after rapid mixing. 1 mL of the microsphere dispersion liquids are respectively sucked by using the disposable syringe with a volume of 1 mL to obtain experimental samples. The experimental samples are respectively frozen and dried, and then the microspheres in the disposable syringes are respectively sucked. A percentage of weight (i.e., M1) of the microspheres in each disposable syringe to total weight (i.e., M0) of the microspheres in each experimental sample is taken as an absorbability rate of the microspheres with a calculation formula of (M1/M0)×100%. Specific results are shown in Table 2.

TABLE 2

| Groups | Absorbability rate % |
|---|---|
| Embodiment 1 | 89 |
| Embodiment 2 | 90 |
| Embodiment 3 | 86 |
| Comparative embodiment 1 | 35 |
| Comparative embodiment 2 | 67 |
| Comparative embodiment 3 | 83 |
| Comparative embodiment 4 | 81 |
| Comparative embodiment 5 | 85 |

As shown in Table 2, absorbability rates of the microspheres prepared in the embodiments 1-3 are much higher than that in the comparative embodiments 1 and 2. In the comparative embodiment 1, the absorbability rate of single PLA microspheres is only 35%. In the comparative embodiment 2, the surfactant is added to prepare the microspheres, and the absorbability rate is nearly doubled. In the embodiments 1-3 and the comparative embodiments 3-4, the macromolecular substances are added, and the absorbability rate is significantly increased.

### Experimental Embodiment 3

Comparison of protein adsorption of the PLA microspheres is as follows.

When a biomaterial is in contact with the physiological environment, it needs good cell adhesion to avoid rejection in the body and serious side effects. An important point in cell adhesion is that a large number of proteins adhere to cell surface, and the protein adsorption is an important driving force of the cell adhesion.

A concentration of bovine serum albumin (BSA) is taken as an abscissa, and an absorbance of the BSA is taken as an ordinate, to draw a BSA growth curve. BAS solution is prepared, and an absorbance of the BSA solution is measured. In each of the embodiments 1-3 and the comparative embodiments 1-5, the BSA solution is used to incubate the PLA microspheres to obtain an incubated solution. After incubating for 5 hours (h), an absorbance of the incubated solution is measured, a concentration of the incubated solution is calculated, and a concentration difference before and after incubation indicates that the BSA is adsorbed by the PLA microspheres. Specific absorption results are shown in Table 3.

TABLE 3

| | Adsorption capacity of BSA (microgram |
|---|---|
| Groups | per gram abbreviated as μg/g) |
| Embodiment 1 | 3600 |
| Embodiment 2 | 3200 |
| Embodiment 3 | 4000 |
| Comparative embodiment 1 | 500 |
| Comparative embodiment 2 | 1200 |
| Comparative embodiment 3 | 2800 |
| Comparative embodiment 4 | 3000 |
| Comparative embodiment 5 | 2800 |

As shown in Table 3, the adsorption capacity of the BSA of the microspheres in the comparative embodiment 1 which is pure PLA microsphere is 500 μg/g. It should be noted that the higher the adsorption capacity of the BSA means the better protein adsorption. In the comparative embodiment 2, the surfactant is added to prepared the microspheres, which improves the protein adsorption of the microspheres. In the comparative embodiments 3-4, the macromolecular substances are added to prepare the microspheres, which significantly improves the protein adsorption of the microspheres. In the embodiments 1-3, the adsorption capacity of the BSA can achieve 3200 μg/g, which shows that the PLA microspheres prepared by the method of the disclosure have excellent cell adhesion, and the PLA microspheres can be used to prepare the drug sustained-release microspheres, to be thereby applied in injection.

Finally, it should be noted that: the above embodiments merely show some of the embodiments of the disclosure, and are not used to limit the disclosure. For those skilled in the art, any modifications, equivalent substitutions and improvements made without departing from a concept of the disclosure shall be included in the scope of protection of the disclosure. Therefore, the scope of protection of the disclosure shall be based on claims.

What is claimed is:

1. A preparation method of injectable polylactic acid (PLA) microspheres, comprising:
S01, preparing a spray solution;
S02, performing homogeneous emulsification on the spray solution to obtain an emulsified spray solution; and
S03, drying the emulsified spray solution to obtain the injectable PLA microspheres.

2. The preparation method of the injectable PLA microspheres as claimed in claim 1, wherein in the step S01, the spray solution comprises an oil phase and an aqueous phase, and a weight ratio of the oil phase and the aqueous phase is 1-10:1-10; and the oil phase comprises PLA and an organic solvent, and the aqueous phase comprises a surfactant, macromolecular substances and deionized water.

3. The preparation method of the injectable PLA microspheres as claimed in claim 2, wherein a molecular weight of the PLA is in a range of 50000-95000.

4. The preparation method of the injectable PLA microspheres as claimed in claim 2, wherein the organic solvent is one selected from the group consisting of dichloromethane, trichloromethane, and ethyl acetate.

5. The preparation method of the injectable PLA microspheres as claimed in claim 2, wherein a concentration of the PLA is in a range of 20-60 grams per liter (g/L).

6. The preparation method of the injectable PLA microspheres as claimed in claim 2, wherein the surfactant is one selected from the group consisting of sodium dodecylbenzene sulfonate, polysorbate 80, and polysorbate 20; and
the macromolecular substances are a mixture of sodium alginate, whey protein and β-glucose, and a weight ratio of the sodium alginate: the whey protein: the β-glucose mixture is 1-6:1-3: 1-5.

7. The preparation method of the injectable PLA microspheres as claimed in claim 2, wherein a concentration of the surfactant is in a range of 5-10 g/L, and a concentration of the macromolecular substances is in a range of 0.5-1 g/L.

8. The preparation method of the injectable PLA microspheres as claimed in claim 1, wherein in the step S02, a temperature of the homogeneous emulsification for the spray solution is 50 Celsius degrees (° C.), a rotational speed of a homogenizer is in a range of 3000-5000 revolutions per minute (rpm), and a time of the homogeneous emulsification for the spray solution is in a range of 5-10 minutes (min).

9. The preparation method of the injectable PLA microspheres as claimed in claim 1, wherein in the step S03, process conditions of the drying the emulsified spray solution comprise: an input rate of the emulsified spray solution being in a range of 4-20 milliliters per minute (mL/min), a pressure of compressed air being in a range of 0.3-0.5 megapascals (MPa), an air inlet temperature being 160° C., an air outlet temperature being 80° C., and carrier gas being inert gas or air; wherein the PLA microspheres are collected in a cyclone separator, and are dried under reduced pressure below 60° C.

10. An application method of the injectable PLA microspheres as claimed in claim 1, comprising:
preparing drug sustained-release microspheres by applying the injectable PLA microspheres.

11. The application method of the injectable PLA microspheres as claimed in claim 10, wherein weight of the drug sustained-release microspheres is in a range of 0.1-10% of weight of the PLA microspheres.

12. A preparation method of injectable PLA microspheres, comprising:
preparing a spray solution; wherein the spray solution comprises an oil phase and an aqueous phase, and a weight ratio of the oil phase and the aqueous phase is 1-10:1-10; and the oil phase comprises PLA and an organic solvent, and the aqueous phase comprises a surfactant, macromolecular substances and deionized water;
performing homogeneous emulsification on the spray solution to obtain an emulsified spray solution; wherein a temperature of the homogeneous emulsification for the spray solution is 50° C., a rotational speed of a homogenizer is in a range of 3000-5000 rpm, and a time of the homogeneous emulsification for the spray solution is in a range of 5-10 min; and
drying the emulsified spray solution to obtain the injectable PLA microspheres; wherein an input rate of the emulsified spray solution is in a range of 4-20 mL/min, a pressure of compressed air is in a range of 0.3-0.5 MPa, an air inlet temperature is 160° C., an air outlet temperature is 80° C., and carrier gas is inert gas or air.

13. The preparation method of the injectable PLA microspheres as claimed in claim 12, wherein a molecular weight of the PLA is in a range of 50000-95000; and a concentration of the PLA is in a range of 20-60 g/L.

14. The preparation method of the injectable PLA microspheres as claimed in claim 12, wherein the organic solvent is one selected from the group consisting of dichloromethane, trichloromethane, and ethyl acetate.

15. The preparation method of the injectable PLA microspheres as claimed in claim 12, wherein the surfactant is one selected from the group consisting of sodium dodecylbenzene sulfonate, polysorbate 80, and polysorbate 20; and
the macromolecular substances are a mixture of sodium alginate, whey protein and β-glucose, and a weight ratio of the sodium alginate: the whey protein: the β-glucose mixture is 1-6:1-3: 1-5.

16. The preparation method of the injectable PLA microspheres as claimed in claim 12, wherein a concentration of the surfactant is in a range of 5-10 g/L, and a concentration of the macromolecular substances is in a range of 0.5-1 g/L.

17. The preparation method of the injectable PLA microspheres as claimed in claim 12, wherein a molecular weight of the PLA is 50000.

18. The preparation method of the injectable PLA microspheres as claimed in claim 17, wherein the weight ratio of the oil phase and the aqueous phase is 5:3; the oil phase comprises the PLA and dichloromethane, the concentration of the PLA is 40 g/L; the aqueous phase comprises polysorbate 20, the macromolecular substances and the deionized water, a concentration of the polysorbate 20 is 5 g/L, and a concentration of the macromolecular substances is 0.5 g/L; and the macromolecular substances are a mixture of sodium alginate, whey protein and β-glucose, and a weight ratio of the sodium alginate: the whey protein: the β-glucose mixture is 2:1:1.

19. The preparation method of the injectable PLA microspheres as claimed in claim 17, wherein the weight ratio of the oil phase and the aqueous phase is 3:5; the oil phase comprises the PLA and dichloromethane, the concentration of the PLA is 50 g/L; the aqueous phase comprises sodium dodecylbenzene sulfonate, the macromolecular substances and the deionized water, a concentration of the sodium dodecylbenzene sulfonate is 5 g/L, and a concentration of the macromolecular substances is 0.5 g/L; and the macromolecular substances are a mixture of sodium alginate, whey protein and β-glucose, and a weight ratio of the sodium alginate: the whey protein: the β-glucose mixture is 3:1:2.

20. The preparation method of the injectable PLA microspheres as claimed in claim 17, wherein the weight ratio of the oil phase and the aqueous phase is 5:5; the oil phase comprises the PLA and dichloromethane, the concentration of the PLA is 40 g/L; the aqueous phase comprises polysorbate 80, the macromolecular substances and the deionized water, a concentration of the polysorbate 80 is 5 g/L, and a concentration of the macromolecular substances is 0.5 g/L; and the macromolecular substances are a mixture of sodium alginate, whey protein and β-glucose, and a weight ratio of the sodium alginate: the whey protein: the β-glucose mixture is 6:3:5.
